(19) United States
(12) Patent Application Publication
(10) Pub. No.: US 2008/0146805 A1
(43) Pub. Date: Jun. 19, 2008

(54) Process for the Production of Levorphanol and Related Compounds

(75) Inventor: Joseph P. Haar, Edwardsville, IL (US)

Correspondence Address: Christy R Cooke;MALLINCKRODT INC, 675 McDonnell Boulevard, Post Office Box 5840, St. Louis, MO, 63134, US

(73) Assignee: MALLINCKRODT INC., St. Louis, MO (US)

(21) Appl. No.: 11/632,254

(22) PCT Filed: Aug. 17, 2005
(86) PCT No.: PCT/US05/29437

§ 371 Date: Jan. 11, 2007

Related U.S. Application Data
(60) Provisional application No. 60/602,195, filed on Aug. 17, 2004.

(51) Int. Cl. C07D 221/28

(52) U.S. Cl. 546/74

(57) ABSTRACT

A process for the production of morphinans with higher purity and yield, when compared to the conventional process, is described. Specifically, the process may be used to prepare levorphanol, levorphanol tartrate, and levorphanol tartrate dihydrate in high yields and substantially free from several process impurities.

### CROSS-REFERENCE TO RELATED APPLICATIONS

This application claims priority to U.S. provisional application No. 60/602,195, filed on Aug. 17, 2004, the contents of which are incorporated herein in their entirety.

### BACKGROUND OF THE INVENTION

Levorphanol (CAS No.: 77-07-6) and levorphanol tartrate (CAS No.: 125-72-4) are well known narcotic opioid analgesics that belong to a class of chemical compounds known as morphinans. Structures of these compounds are shown next.

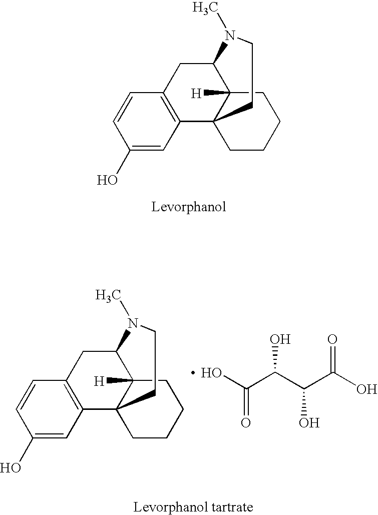

Levorphanol and levorphanol tartrate are conventionally prepared from 3-methoxy-N-methylmorphinan hydrobromide. 3-Methoxy-N-methylmorphinan hydrobromide is reacted with aqueous hydrobromic acid to replace the methoxy group with a hydroxyl. 3-hydroxy-N-methylmorphinan hydrobromide is neutralized with ammonium hydroxide to form crude levorphanol. The crude levorphanol formed can be converted to anhydrous levorphanol or reacted with aqueous tartaric acid to form levorphanol tartrate and levorphanol tartrate dihydrate (CAS No.: 5985-38-6).

One of many challenges with the conventional production process is to minimize or eliminate the presence of impurities. As levorphanol, levorphanol tartrate, and levorphanol tartrate dihydrate are prescribed as drugs, there is a need to produce them in forms that contain minimal levels of impurities.

Therefore there is a need in the art to develop new processes that reduce or eliminate the undesirable impurities produced by the conventional processes used to synthesize levorphanol, levorphanol tartrate, and levorphanol tartrate dihydrate.

### SUMMARY OF THE INVENTION

One aspect of the invention is directed to a process for the synthesis of morphinans and structurally related compounds.

Another aspect of the invention is directed to an improved process for the production of levorphanol, levorphanol tartrate, or levorphanol tartrate dihydrate.

Yet another aspect of the invention is directed to a process for removing impurities from levorphanol, levorphanol tartrate or levorphanol tartrate dihydrate.

An additional aspect of the invention is directed to levorphanol, levorphanol tartrate, or levorphanol tartrate dihydrate in which the amount of impurities is reduced.

Other aspects, features, and advantages of the invention will become apparent from the following detailed description and the figures.

### BRIEF DESCRIPTION OF THE FIGURES

FIG. 1 shows the conventional process for the production of levorphanol, levorphanol tartrate, and levorphanol tartrate dihydrate.

FIG. 2 shows the process for the production of levorphanol, levorphanol tartrate, and levorphanol tartrate dihydrate according to the present invention.

### DETAILED DESCRIPTION

The conventional process for the production of levorphanol, levorphanol tartrate, and levorphanol tartrate dihydrate starts with 3-methoxy-N-methylmorphinan. 3-Methoxy-N-methylmorphinan hydrobromide is reacted with aqueous an aqueous HBr solution containing less than 50% HBr. This reaction replaces the 3-methoxy group with a 3-hydroxy group, producing levorphanol in a solution of HBr. This solution is neutralized using NH₄OH and extracted with a mixture of chloroform and isopropanol. The levorphanol ends up in the organic layer and can be extracted. If levorphanol tartrate is desired, crude solid levorphanol is dissolved in isopropanol and tartaric acid is added. Crystallization of levorphanol tartrate from 66% aqueous isopropanol produces levorphanol tartrate dihydrate.

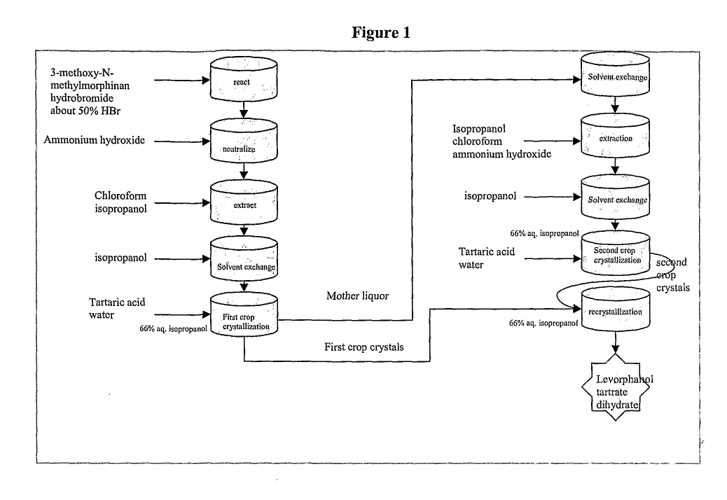

One major problem with the conventional process is that the yield of levorphanol tartrate dihydrate is low (less than 60%). Therefore, the conventional process included a recovery of the product from first crop mother liquor. A second crop of the product is prepared, isolated and combined with the first crop crystals and recrystallized to yield the final product (FIG. 1).

Another major problem was that the conventional processes produced final products that contained several undesirable impurities. Once these impurities are generated in the conventional synthetic processes, they became incorporated into the final products. Standard recrystallization processes fail to remove these impurities from the final products.

In summary, the conventional process is low yielding and produces products with undesirable impurities.

The process according to the present invention produces product of higher quality as determined by chromatographic purity and assay. Further, the two-step crystallization of the present invention effectively removes several process impurities including 2-bomolevorphanol and 10-ketolevorphanol. Finally, the process according to the present invention, including sequential extractions with a water soluble amine base, improves the product yield. In other words, the process of the present invention produces purer products in higher yields.

One example of the process according to the present invention starts with a mixture of 3-methoxy-N-methylmorphinan hydrobromide in an aqueous solution of a halogen acid to form 3-hydroxy-N-methylmorphinan hydrobromide. Examples include HF, HCl, HBr, and HI. The concentration of the acid in water can range from 5% to 95%, preferably 25% to 75%, and most preferably about 50%. In a preferred embodiment, 48% HBr is used.

The mixture is optionally heated, preferably to reflux, and thereafter, preferably cooled to a temperature less than room temperature, more preferably to a temperature of about 20° C. A mixture of water, an amine base, preferably ammonium hydroxide, a halogenated solvent, preferably chloroform, and a lower alcohol, preferably isopropanol is combined and the resulting mixture allowed to settle into two layers. After extraction of the organic layer with an amine base solution, preferably ammonium hydroxide in water, a lower alcohol, preferably isopropanol is combined and the resulting mixture is preferably heated.

In one embodiment of the present invention, the process includes more than one sequential extraction of the organic layer with a water soluble amine base, preferably ammonium hydroxide, to increase yield. The extraction of the organic layer removes excess bromide ions from the organic layer. In a preferred embodiment, the organic layer is extracted with a water soluble amine base for 2 to 5 times, more preferably, 3 or 4 times, most preferred, 4 times.

A solution of tartaric acid is added and levorphanol tartrate crystallizes out. The solution is preferably heated, more preferably to a temperature between about 35° C. and about 65° C., most preferably between about 40° C. and 50° C. In one embodiment, the crystallization of levorphanol tartrate occurs in an aqueous-organic solvent mixture. An embodiment of the present invention uses a solvent mixture of about 80% to about 100% aqueous isopropanol, preferably 85% to 98% aqueous isopropanol, more preferably about 88% to about 95% aqueous isopropanol, most preferably about 95% aqueous isopropanol. In another embodiment, the product of this crystallization is a substantially anhydrous, preferably a completely anhydrous, levorphanol tartrate salt crystals.

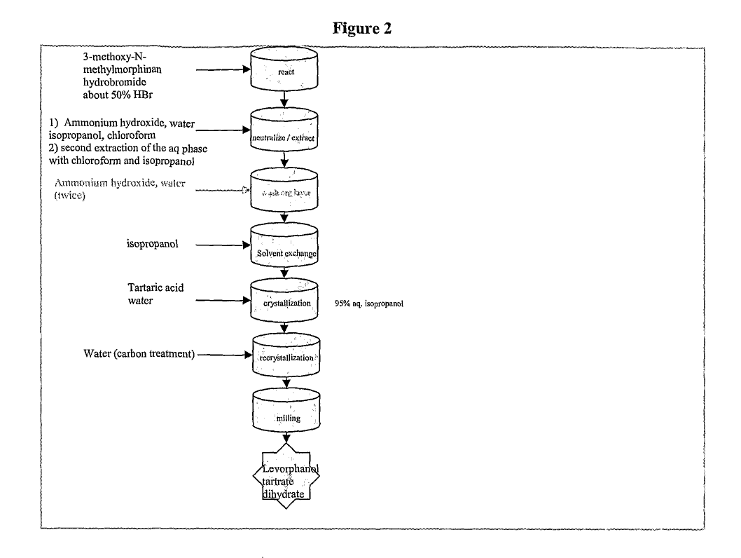

If the desired product is levorphanol tartrate dihydrate, the levorphanol tartrate crystals are hydrated. In one embodiment of the present invention, the levorphanol tartrate wet cake is dried before hydration. The levorphanol tartrate may be dried by passing air over the crystals, in an oven, or by any other techniques known to remove solvent from a solid. To hydrate the levorphanol tartrate, it is suspended in a solvent containing water. The resulting mixture is heated to dissolve the levorphanol tartrate, preferably to a temperature range from 50° C. to about 110° C., more preferably from about 65° C. to about 95° C., most preferably to about 80° C. In a preferred embodiment, charcoal added to the solution and stirred for a time period from about 5 minutes to about 60 minutes, preferably from about 15 minutes to about 45 minutes. The mixture is cooled, preferably to a temperature range from 30° C. to about 75° C., more preferably from about 45° C. to about 65° C., most preferably about 60° C. In a preferred embodiment, the mixture is subjected to a second cooling preferably to a temperature range from −10° C. to about 25° C., more preferably from about −5° C. to about 15° C., most preferably from about 0° C. to 5° C. The resulting crystals of levorphanol tartrate dihydrate may be dried (FIG. 2).

The product produced was analyzed using a variety of techniques including X-Ray Diffraction (XRD), Microscopy (MICR), Scanning Electron Microscopy (SEM), Infrared (IR), Thermal Gravimetric Analysis (TGA), Differential Scanning Calorimetry (DSC), and Particle Size Analysis (PTSZ). All these techniques indicate that the morphology of the product produced by the present invention is similar to that produced by the conventional process.

Any water soluble organic solvent may be used for the crystallizations of levorphanol, levorphanol tartrate, or levorphanol tartrate dihydrate including acetonitrile, acetone and other water soluble ketones, water soluble alcohols, THF and other water soluble ethers, diglyme and other glymes, and mixtures of the same. Examples of suitable alcohols include methyl alcohol, ethyl alcohol, n-propyl alcohol, n-butyl alcohol, iso-butyl alcohol, tertiary butyl alcohol, n-pentyl alcohol, iso-pentyl alcohol, and neo-pentyl alcohol. Preferably the alcohol used as a solution in water, in which the concentration of alcohol is greater than 80% (w/w). The recrystallization of levorphanol tartrate dihydrate from the anhydrous form is preferably conducted in water. However, other solvents or solvent mixtures may be used as long as they yield the product with the desired purity, yield and degree of hydration. For example, a water and alcohol mixture at a concentration of about 75% or less alcohol may be used for the crystallization of levorphanol tartrate dihydrate.

The process of the present invention may be used to produce any morphinan or structurally-related classes of compounds. Preferably, the process is used to produce at least one of the following compounds: levorphanol, levorphanol tartrate, or levorphanol tartrate dihydrate. Most preferably, the process is used to produce levorphanol tartrate dihydrate.

### EXAMPLE 1

### Production of Levorphanol Tartrate Dihydrate

A flask was charged with solid 3-methoxy-N-methylmorphinan hydrobromide (52.0 g). To the solid, aqueous 48% HBr (2.91 g/g, 151 g) was added. The mixture was heated to reflux (about 125° C.), under a continuous nitrogen purge of the vapor space, for 2.5 hours. The reaction was then cooled to 20° C. and added to a mixture of water (1.71 g/g, 88.9 g), ammonium hydroxide (30%, 1.12 g/g, 58.2 g), chloroform (3.81 g/g, 198 g), and isopropanol (0.66 g/g, 34 g) at a rate that kept the temperature between 20° C. and 30° C. Once the addition was complete, the mixture was stirred for 15 minutes. The mixture was allowed to settle into two layers.

The organic layer (bottom layer) was extracted twice with a solution of NH₄OH (5.23 M, 1.37 g/g, 71.2 ml). Isopropanol was then added to the resulting organic layer to facilitate heat transfer and to keep solid levorphanol from forming in the reactor. During the distillation, more isopropanol was added to maintain a solution and facilitate solvent exchange. The mixture was distilled until the temperature of the solution equaled the boiling point of isopropanol (about 82.2° C.). The solution was cooled and assayed for levorphanol via HPLC. The target concentration of levorphanol was 8.27% w/w. Either more isopropanol was added to reach this concentration or more was distilled until the amount of levorphanol was greater than 8.27% w/w. Once this amount was reached, the temperature of the mixture was brought to 60° C.

A solution of 50% tartaric acid was prepared from tartaric acid (0.427 g/g, 22.2 g) and water (0.427 g/g, 22.2 g) and warmed to a temperature between about 40° C. and about 50° C. The warm tartaric acid solution was added to the levorphanol/isopropanol mixture. After the addition is complete, the mixture is stirred for about 15 minutes and then heated to 75° C. and held at that temperature for between about 30 and about 60 minutes.

Crystallization of levorphanol tartrate began within a few minutes of the addition of the warm aqueous tartaric acid solution and the crystallization reaction was slightly exothermic. Holding the mixture at a temperature of about 60° C. for a few minutes kept the warm mixture from refluxing while the crystallization began. Cooling to 0° C. to 5° C. and holding in this range for at least 60 minutes completed the crystallization process. The crystals were filtered and dried on the filter for about 1 to about 2 hours.

The crystals produced may contain water. In a preferred embodiment of the present invention, the levorphanol tartrate crystals do not contain any water. If the crystals are not dry, the overall product yield suffers.

The solid anhydrous levorphanol tartrate (60.1 g) was suspended in water (2.25 g/g, 135 g). The resulting mixture was heated to about 80° C. under nitrogen, which resulted in a dissolution of the levorphanol tartrate. Charcoal (20 g/kg, 1.2 g) and filter aid (10 g/kg, 0.6 g) were added to the hot solution, stirred for about 25 minutes, filtered into another flask under nitrogen, and cooled to about 60° C. over 30 minutes. Under these conditions, crystallization of levorphanol tartrate dihydrate occurred. The solution was cooled further to 0° C. to 5° C. and held there for at least 60 minutes. The crystals were collected via vacuum filtration, rinsed with water (0.25 g/g, 15 g, less than 5° C.), and dried overnight at 50° C.

An important step in the purification of levorphanol tartrate and levorphanol tartrate dihydrate is recrystallization from 95% (w/w) aqueous isopropanol, though any alcohol at a sufficiently high concentration may be used. The crude levorphanol tartrate dihydrate produced above is preferably dissolved in 95% (w/w) aqueous isopropanol, though any aqueous-organic solvent mixture in which the organic component is present in an amount greater than 80% (w/w) may be used. The resulting solid is the anhydrous salt of levorphanol tartrate, which can be isolated and processed.

In a preferred embodiment, the anhydrous levorphanol tartrate is subsequently hydrated to the dihydrate salt by dissolving it in hot water followed by a second recrystallization upon cooling to about 1° C. to 5° C. Levorphanol tartrate dihydrate is dissolved in hot 95% (w/w) aqueous isopropanol. Crystals of nearly anhydrous levorphanol tartrate precipitated. The crystals were collected by filtration and recrystallized from water to generate levorphanol tartrate dihydrate. The anhydrous assay (HPLC test) was 100.7% and the chromatographic purity was 99.75%. The area percent for 10-ketolevorphanol and 2-bromolevorphanol were 0.06% and 0.05% respectively.

### EXAMPLE 2

### Removal of Excess Bromide Ions

A mass balance analysis for bromide ion (Br⁻) was conducted to determine its fate in the synthetic process. Ammonium hydroxide was used in both the conventional process and in the process according to the present invention. Its purpose was to react with HBr to produce NH₄Br, which is expected to remain in the aqueous layer. In contrast, levorphanol is expected to be in base form in the organic layer.

Levorphanol was found to react with HBr and NH₄Br to form levorphanol hydrobromide, which is soluble in water. Therefore, if there are any bromide ions in the organic layer, they will react with levorphanol to form levorphanol hydrobromide. As a result, the yield of the overall reaction will be reduced.

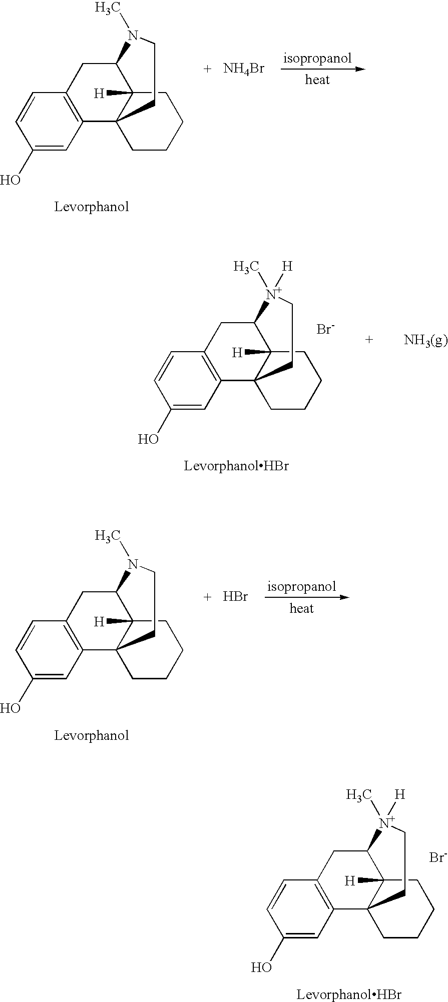

To test this hypothesis, the aqueous layer from the addition of the mixture of water, ammonium hydroxide, chloroform, and isopropanol was analyzed and found to contain approximately 84% of the Br⁻ in it while the organic layer had approximately 16% of the Br⁻. This bromide assay clearly established that levorphanol hydrobromide was soluble in the organic layer. The molar ratio of HBr to levorphanol just prior to neutralization was 6.07:1. Since 16% of this Br⁻ was in the organic layer, the molar ratio of Br⁻ to levorphanol in the organic layer was 0.971:1. In other words, nearly a full equivalent of Br⁻ was in the presence of levorphanol. Because levorphanol hydrobromide was soluble in aqueous isopropanol and because nearly a full equivalent of Br⁻ was in the organic layer, the significant bromide ion contamination of the organic layers led to lower yields of levorphanol tartrate.

The organic layer from the mass balance experiment, which was extracted twice with aqueous NH₄OH contained 95% of the Br⁻. After a third extraction, the combined aqueous phases contained 97% of the Br⁻. It was clear that multiple extractions of the organic layer were required in order to reduce the Br⁻ content of the organic layer and improve the yield of anhydrous levorphanol tartrate. This was confirmed in a subsequent experiment in which one portion of the levorphanol reaction mixture (levorphanol in 48% HBr) was worked up as described (i.e., with two NH₄OH washes) and compared with another portion of the levorphanol reaction mixture in which two additional aqueous NH₄OH extractions were performed on the organic layers before the solvent exchanges were achieved. The second portion, with the additional NH₄OH extractions had higher yield (91%) of anhydrous levorphanol tartrate compared to the first portion (88%).

### EXAMPLE 3

### Identification and Isolation of a Process Impurity

Examination of batches of levorphanol tartrate revealed an impurity at a level greater than 0.1% (w/w). The impurity was analyzed and found to be the methyl quaternary ammonium salt of levorphanol. This salt resulted from the reaction of some of the methyl bromide produced as a byproduct in the reaction with levorphanol upon work up.

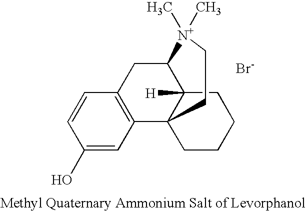

Batches of levorphanol tartrate having some level of quaternary salt contamination were reprocessed according to the double crystallization procedure (using isopropanol first, followed by water) developed for the removal of 2-bromolevorphanol and 10-ketolevorphanol. It was found that this procedure also reduced the level the quaternary salt from the product. The Chromatographic Purity of the product after the double recrystallization procedure ranged from 99.68% to 99.75%. This was excellent quality material with all known impurities far below their limits. In addition, the two most undesirable impurities, 2-bromolevorphanol and 10-ketolevorphanol, were not detected in the product.

The product produced by the process of the present application has the same crystalline form and is higher purity than the product produced by the conventional process. In other words, the new process produced superior levorphanol tartrate dihydrate through more efficient and robust processing. Furthermore, the double crystallization procedure as described is useful for removing 10-ketolevorphanol, 2-bromolevorphanol, and N-methyllevorphanol quaternary salt.

It is to be understood that while the invention has been described above using specific embodiments, the description and examples are intended to illustrate the structural and functional principles of the present invention and are not intended to limit the scope of the invention. On the contrary, the present invention is intended to encompass all modifications, alterations, and substitutions.

1. A process for purifying a morphinan, or a salt thereof, comprising:
forming a biphasic mixture comprising ammonium hydroxide, chloroform, isopropanol, and water, wherein the biphasic mixture comprises an aqueous layer and an organic layer,
dissolving the morphinan in the biphasic mixture,
extracting the organic layer including the morphinan with a water soluble amine base,
isolating the morphinan,
forming a salt of the morphinan,
crystallizing the morphinan salt in an aqueous-organic solvent mixture, wherein the organic solvent in the aqueous-organic solvent mixture is in a concentration of about 85% (w/w) to about 100% (w/w), and
optionally, recovering the morphinan from the salt.

2. The process of claim 1, wherein the organic solvent is in a concentration of from about 85% (w/w) to about 98% (w/w).

3. The process of claim 2, wherein the organic solvent is in a concentration of from about 88% (w/w) to about 9:5% (w/w).

4. The process of claim 3, wherein the organic solvent is in a concentration of about 95% (w/w).

5. The process of claim 1, wherein the organic solvent is selected from the group consisting of a water soluble nitrile, a water soluble ketone, a water soluble alcohol, a water soluble ether, a glyme, and mixtures thereof.

6. The process of claim 5, wherein the water soluble nitrile is acetonitrile.

7. The process of claim 5, wherein the water soluble ketone is acetone.

8. The process of claim 5, wherein the water soluble alcohol is selected from at least one of methyl alcohol, ethyl alcohol, and iso-propyl alcohol.

9. The process of claim 8, wherein the water soluble alcohol is iso-propyl alcohol.

10. The process of claim 5, wherein the water soluble ether is THF.

11. The process of claim 5, wherein the water soluble glyme is diglyme.

12. The process of claim 1, wherein the morphinan is levorphanol.

14. A process for purifying a morphinan salt, comprising:
dissolving a hydrated salt of the morphinan in an aqueous-organic solvent mixture, wherein the organic solvent in the aqueous-organic solvent mixture is in a concentration of about 85% (w/w) to about 100% (w/w),
crystallizing the morphinan salt, wherein the morphinan salt is substantially anhydrous,
dissolving the substantially anhydrous morphinan salt in an aqueous solvent, and
crystallizing the hydrated salt of the morphinan.

15. The process of claim 14, further comprising drying the substantially anhydrous morphinan salt before dissolving in the aqueous solvent.

16. A process for purifying levorphanol tartrate dihydrate comprising:
dissolving levorphanol tartrate dihydrate in about 95% (w/w) iso-propyl alcohol
crystallizing anhydrous levorphanol tartrate;
dissolving the anhydrous levorphanol tartrate in water; and
crystallizing levorphanol tartrate dihydrate.

21. A method of synthesizing levorphanol comprising:
reacting 3-Methoxy-N-methylmorphinan hydrobromide with aqueous hydrobromic acid to produce aqueous levorphanol hydrobromide;
neutralizing the levorphanol hydrobromide in a biphasic mixture comprising ammonium hydroxide, chloroform, isopropanol, and water, wherein the biphasic mixture comprises an aqueous layer and an organic layer of levorphanol and levorphanol hydrobromide;
extracting the organic layer with a water soluble amine base at least two times;
isolating levorphanol.

22. The method of claim 21, wherein the extraction of the organic layer removes excess bromide ions from the organic layer.

23. The method of claim 21, wherein the water soluble amine base comprises ammonium hydroxide.

24. The method of claim 21, wherein the organic layer is extracted with a water soluble amine base for 2 to 5 times.

25. The method of claim 24, wherein the organic layer is extracted with a water soluble amine base 3 or 4 times.

26. The method of claim 25, wherein the organic layer is extracted with a water soluble amine base 4 times.

27. A method of synthesizing levorphanol tartrate comprising;
reacting 3-Methoxy-N-methylmorphinan hydrobromide with aqueous hydrobromic acid to produce aqueous levorphanol hydrobromide;
neutralizing the levorphanol hydrobromide in a biphasic mixture comprising ammonium hydroxide, chloroform, isopropanol, and water, wherein the biphasic mixture comprises an aqueous layer and an organic layer of levorphanol and levorphanol hydrobromide;
extracting the organic layer with a water soluble amine base at least two times;
adding tartaric acid to the organic layer to form levorphanol tartrate,
isolating levorphanol tartrate; and
crystallizing the levorphanol tartrate from an aqueous-organic solvent mixture, wherein the organic solvent in the aqueous-organic solvent mixture is in a concentration of about 85% to about 100% aqueous organic solvent.

28. A method of synthesizing levorphanol tartrate dihydrate comprising:
reacting 3-Methoxy-N-methylmorphinan hydrobromide with aqueous hydrobromic acid to produce aqueous levorphanol hydrobromide,
neutralizing the levorphanol hydrobromide in a biphasic mixture comprising ammonium hydroxide, chloroform, isopropanol, and water, wherein the biphasic mixture comprises an aqueous layer and an organic layer of levorphanol and levorphanol hydrobromide;
extracting the organic layer with a water soluble amine base at least two times;
adding tartaric acid to the organic layer to form levorphanol tartrate;
isolating levorphanol tartrate;
crystallizing the levorphanol tartrate from an aqueous-organic solvent mixture, wherein the organic solvent in the aqueous-organic solvent mixture is in a concentration of about 85% to about 100% aqueous organic solvent;
isolating anhydrous levorphanol tartrate;
dissolving anhydrous levorphanol tartrate in an aqueous solvent; and
crystallizing levorphanol tartrate dihydrate from the aqueous solvent.

29. A process for purifying a morphinan, or a salt thereof, comprising:
dissolving a salt or hydrated salt of the morphinan in aqueous iso-propyl alcohol;
wherein the iso-propyl alcohol is in a concentration of about 85% (w/w) to about 98% (w/w),
crystallizing the morphinan salt, wherein the morphinan salt is substantially anhydrous; and
optionally, recovering the morphinan from the salt.

30. The process of claim 29, wherein the morphinan is levorphanol.

31. The process of claim 29, wherein the hydrated salt of the morphinan is levorphanol tartrate dihydrate.

32. The process of claim 29, wherein the morphinan salt is levorphanol tartrate.

33. The process of claim 14, wherein the aqueous solvent is water.

34. The process of claim 28, wherein the aqueous solvent is water.
